(19) United States
(12) Patent Application Publication
(10) Pub. No.: US 2009/0159874 A1
(43) Pub. Date: Jun. 25, 2009

(54) Organic electroluminescent devices

(76) Inventors: Horst Vestweber, Gilersberg-Winterscheid (DE); Philipp Stoessel, Frankfurt am Main (DE); Anja Gerhard, Darmstadt (DE); Amir Parham, Frankfurt (DE)

Correspondence Address: CONNOLLY BOVE LODGE & HUTZ, LLP, P O BOX 2207, WILMINGTON, DE, 19899, US

(21) Appl. No.: 11/630,637

(22) PCT Filed: Jun. 22, 2005
(86) PCT No.: PCT/EP2005/06727

§ 371 Date: Jan. 30, 2007

(30) Foreign Application Priority Data
Jun. 26, 2004 (DE) 10 2004 031 000.9

(51) Int. Cl. H01L 51/00; C07C 211/54

(52) U.S. Cl. 257/40; 564/322; 257/E51.001

(57) ABSTRACT

The present invention relates to the improvement of organic electroluminescent devices, in particular blue-emitting devices, by using compounds of the formula (1)

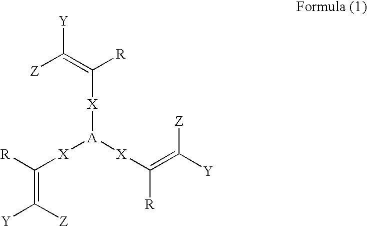

as dopants in the emitting layer.

The present invention describes the use of certain compounds in organic electroluminescent devices.

The use of semiconducting organic compounds which are capable of the emission of light in the visible spectral region in organic electroluminescent devices (OLEDs) is just at the beginning of the market introduction. The general structure of organic electroluminescent devices is described, for example, in U.S. Pat. No. 4,539,507, U.S. Pat. No. 5,151,629, EP 0676461 and WO 98/27136. For simple devices containing OLEDs, the market introduction has already taken place, as confirmed by the car radios from Pioneer, the mobile telephones from Pioneer and SNMD or a digital camera from Kodak with an “organic display”. Further products of this type are just about to be introduced.

However, these devices still exhibit considerable problems which require urgent improvement:
1. The operating lifetime is still short, in particular in the case of blue emission, meaning that it has to date only been possible to achieve simple applications commercially.
2. The efficiency has been improved in recent years, but is still too low, especially in the case of fluorescent OLEDs, and must be improved.
3. The operating voltage is fairly high, especially in the case of fluorescent OLEDs, and should therefore be reduced further in order to improve the power efficiency. This is of major importance, in particular, for mobile applications.
4. Many blue-emitting emitters which comprise both aromatic amines and also double-bond systems are thermally unstable and decompose during sublimation or during vapour deposition. The use of these systems is consequently impossible or only possible with considerable losses and with high technical complexity.

As closest prior art, mention may be made of the use of certain arylvinylamines which are unsubstituted at the double bond of the stilbene-like system by Idemitsu (for example WO 04/013073, WO 04/016575, WO 04/018587). Very good lifetimes in the case of dark-blue emission are thus quoted. However, these results are highly dependent on the host material used, meaning that the quoted lifetimes cannot be compared as absolute values, but always only on use in an optimised system. Furthermore, these compounds are thermally unstable and cannot be evaporated without decomposition, which requires high technical complexity for the vapour deposition and thus represents a significant industrial disadvantage. Without wishing to be tied to a certain theory, we assume that the thermal instability is at least partly due to the fact that the compounds can exhibit thermal cis/trans isomerisation. A further disadvantage is the emission colour of these compounds. While Idemitsu quotes dark-blue emission (CIE y coordinates in the range 0.15-0.18), it has not been possible to reproduce these colour coordinates in simple devices in accordance with the prior art. On the contrary, green-blue emission is obtained here. It is not evident how blue emission can be produced using these compounds.

The use of aryl-substituted tristilbenamines in OLEDs has already been described in the literature:

JP 10-255979 describes the use of various unsubstituted and substituted arylvinylamines in the hole-injection layer of OLEDs. The use of these compounds as emitting materials is not evident from the description.

JP 2913116 describes the use of unsubstituted and substituted tristilbenamines and other arylvinyl compounds in OLEDs. These compounds are employed here as the pure substance in the emitting layer. Although it is described that these devices “light up attractively”, the absence of quantitative device data indicates, however, that efficiency, operating voltage, lifetime and colour are not satisfactory. Furthermore, it is described that each carbon atom of the double bond is preferably only monosubstituted and that thus, for example, simple stilbene derivatives are preferred over triarylethene derivatives. It is not evident from the description that aryl-substituted tristilbenamine derivatives could be particularly and more suitable than unsubstituted derivatives for use in OLEDs. In particular, it is not evident in what form or in what device structure apart from that described (use as the pure substance in the emitting layer) such compounds could particularly usefully be employed.

Thus, there continues to be a demand for blue-emitting compounds which result in good efficiencies and at the same time in long lifetimes in organic electroluminescent devices and which can be processed industrially without problems. Surprisingly, it has now been found that organic electroluminescent devices which comprise certain compounds—shown below—as blue-emitting dopants in a host material have significant improvements over the prior art. Using these materials, it is possible at the same time to obtain high efficiencies and long lifetimes. In addition, these compounds, in contrast to materials in accordance with the prior art, can be sub-limed and vapour-deposited without significant decomposition and are therefore significantly easier to handle than materials in accordance with the prior art.

The invention relates to organic electroluminescent devices comprising cathode, anode and at least one emitting layer, characterised in that the emitting layer comprises

a) 0.1 to 99% by weight of at least one compound of the formula (1)

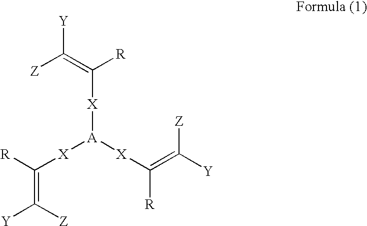

where the following applies to the symbols and indices:
A is on each occurrence nitrogen, phosphorus, P═O, P═S, arsenic, As═O, As═S, antimony, Sb═O or Sb═S;
X is on each occurrence, identically or differently, a divalent aromatic or heteroaromatic ring system having 2 to 60 C atoms, which may be substituted by one or more radicals R¹;
Y is on each occurrence, identically or differently, a monovalent aromatic or heteroaromatic ring system having 2 to 60 C atoms, which may be substituted by one or more radicals R¹, with the proviso that Y cannot contain substituted or unsubstituted amino groups;
Z is on each occurrence, identically or differently, Y or CN or a straight-chain, branched or cyclic alkyl group having 1 to 40 C atoms, which may be substituted by one or more radicals R², where one or more non-adjacent CH₂ groups may be replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms may be replaced by F, Cl, Br, I, CN or NO₂;
R is on each occurrence, identically or differently, H, CN or a straight-chain, branched or cyclic alkyl group having 1 to 40 C atoms, which may be substituted by one or more radicals R², where one or more non-adjacent CH₂ groups may be replaced by —R²C═CR²—, —C═C—, Si(R²)₂, Ge(R²)₂, Sn(R²)₂, C═O, C═S, C═Se, C═NR², —O—, —S— or —CONR²— and where one or more H atoms may be replaced by F, Cl, Br, I, CN or NO₂;
R¹ is on each occurrence, identically or differently, H, F, Cl, Br, I, CN, NO₂, a straight-chain, branched or cyclic alkyl, alkoxy or thioalkoxy group having 1 to 40 C atoms, which may be substituted by one or more radicals R², where one or more non-adjacent CH₂ groups may be replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms may be replaced by F, Cl, Br, I, CN or NO₂, or an aromatic or heteroaromatic ring system, which may be substituted by one or more radicals R², or an aryloxy or heteroaryloxy group having 1 to 40 aromatic C atoms, which may be substituted by one or more radicals R², or a combination of two to five of these systems; two or more substituents R² here may also form a mono- or polycyclic, aliphatic or aromatic ring system with one another; with the proviso that R¹ does not represent a cyclohexylidenemethine group;
R² is on each occurrence, identically or differently, H or an aliphatic or aromatic hydrocarbon radical having 1 to 20 C atoms; and
   b) 1 to 99.9% by weight of at least one host material.

For the purposes of this invention, an aromatic ring system contains 6 to 60 C atoms. For the purposes of this invention, a heteroaromatic ring system contains at least two C atoms and at least one heteroatom, preferably selected from nitrogen, sulfur and/or oxygen, with the proviso that the total number of C atoms and hetero-atoms is at least 5. For the purposes of this invention, an aromatic or heteroaromatic ring system is intended to be taken to mean a system which does not necessarily contain only aromatic or heteroaromatic groups, but instead in which a plurality of aromatic or heteroaromatic groups may also be interrupted by a short, non-aromatic unit (less than 10% of the atoms other than H, preferably less than 5% of the atoms other than H), such as, for example, an sp³-hybridised C, N or O atom. Thus, for example, systems such as 9,9′-spirobifluorene, 9,9-diarylfluorene, triarylamine, diaryl ether, etc., are also intended to be taken to mean aromatic ring systems for the purposes of this application.

The group X is preferably selected in such a way that the conjugation between the central atom A and the double bond is not interrupted.

For the purposes of the present invention, a C₁- to C₄₀-alkyl group, in which, in addition, individual H atoms or CH₂ groups may be substituted by the above-mentioned groups, is particularly preferably taken to mean the radicals methyl, ethyl, n-propyl, i-propyl, n-butyl, i-butyl, s-butyl, t-butyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. A C₁- to C₄₀-alkoxy group is particularly preferably taken to mean methoxy, ethoxy, n-propoxy, i-propoxy, n-butoxy, i-butoxy, s-butoxy, t-butoxy or 2-methylbutoxy. An aromatic or heteroaromatic ring system having 2 to 60 C atoms, which may also in each case be substituted by the above-mentioned radicals R¹ and which may be linked to the aromatic or heteroaromatic ring system via any desired positions, is taken to mean, in particular, groups derived from benzene, naphthalene, anthracene, phenanthrene, pyrene, chrysene, perylene, fluoranthene, naphthacene, pentacene, benzopyrene, biphenyl, biphenylene, terphenyl, terphenylene, fluorene, spirobifluorene, diphenyl ether, triphenylamine, dihydrophenanthrene, dihydropyrene, tetrahydropyrene, cis- or trans-indenofluorene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

Preference is given to organic electroluminescent devices in which the symbol A on each occurrence stands for nitrogen, phosphorus or P═O, particularly preferably for nitrogen.

Preference is furthermore given to organic electroluminescent devices in which the symbol X on each occurrence, identically or differently, stands for a divalent aryl or heteroaryl group having 3 to 40 C atoms, which may be substituted by one to four radicals R¹; X on each occurrence, identically or differently, particularly preferably stands for a divalent aryl group having 6 to 20 C atoms, which may be substituted by one or two radicals R¹.

Preference is furthermore given to organic electroluminescent devices in which the symbol Y, identically or differently on each occurrence, stands for a monovalent aryl or heteroaryl group having 3 to 40 C atoms, which may be substituted by one to four radicals R¹; Y on each occurrence, identically or differently, particularly preferably stands for a monovalent aryl group having 6 to 20 C atoms, which may be substituted by one or two radicals R¹.

Preference is furthermore given to organic electroluminescent devices in which the symbol Z, identically or differently on each occurrence, stands for Y.

Preference is furthermore given to organic electroluminescent devices in which the symbol R, identically or differently on each occurrence, stands for H or a methyl group, particularly preferably for H.

Preference is furthermore given to compounds of the formula (1) in which Y and Z, which are bonded to an alkene group, stand for the same monovalent aromatic ring system and are preferably also identically substituted.

Particular preference is given to compounds of the formula (1) in which all units Y and Z are selected identically.

Preference is again furthermore given to compounds of the formula (1) in which all units X are selected identically.

Particular preference is given to compounds of the formula (1) which have a symmetrical structure and a three-fold axis of rotation (C3), in particular those in which all units X and all units Y and Z and all substituents R, R¹ and R² are in each case selected identically.

In a further preferred embodiment of the invention, the symbols Y and Z each, identically or differently on each occurrence, stand for an aryl or heteroaryl group having 2 to 40 C atoms, which may be substituted by R¹, and form a ring system with one another, either via a substituent R¹ or via a direct bond. Y and Z particularly preferably stand for the same aryl or heteroaryl group, very particularly preferably for benzene. Y and Z are furthermore preferably bonded via a direct bond or a bridge R¹ having 1 to 3 bridge atoms.

Examples of preferred compounds of the formula (1) are Examples 1 to 33 depicted below, which may be substituted by R¹ or unsubstituted.

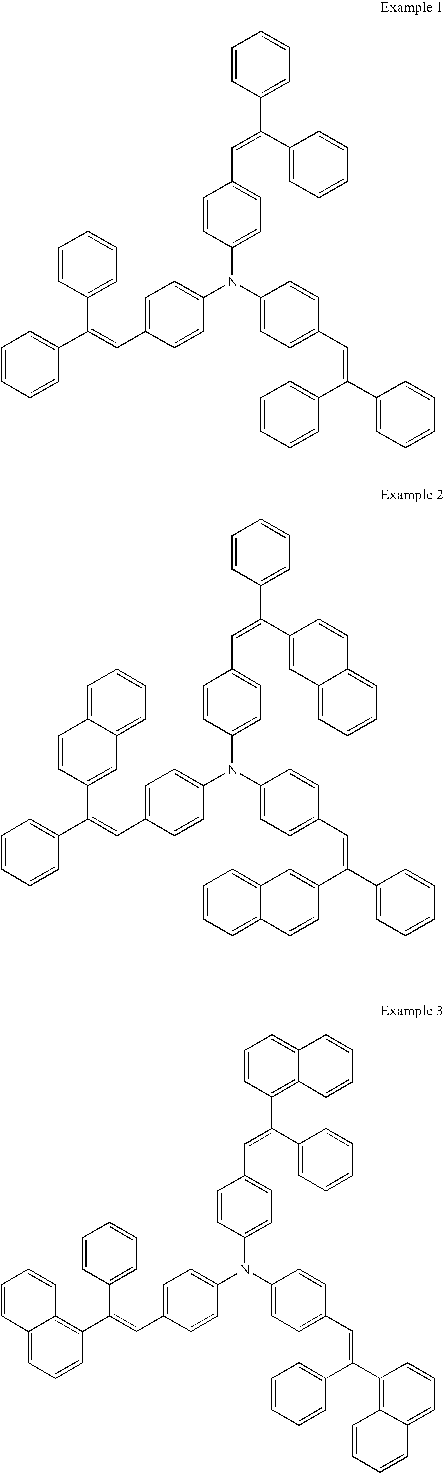

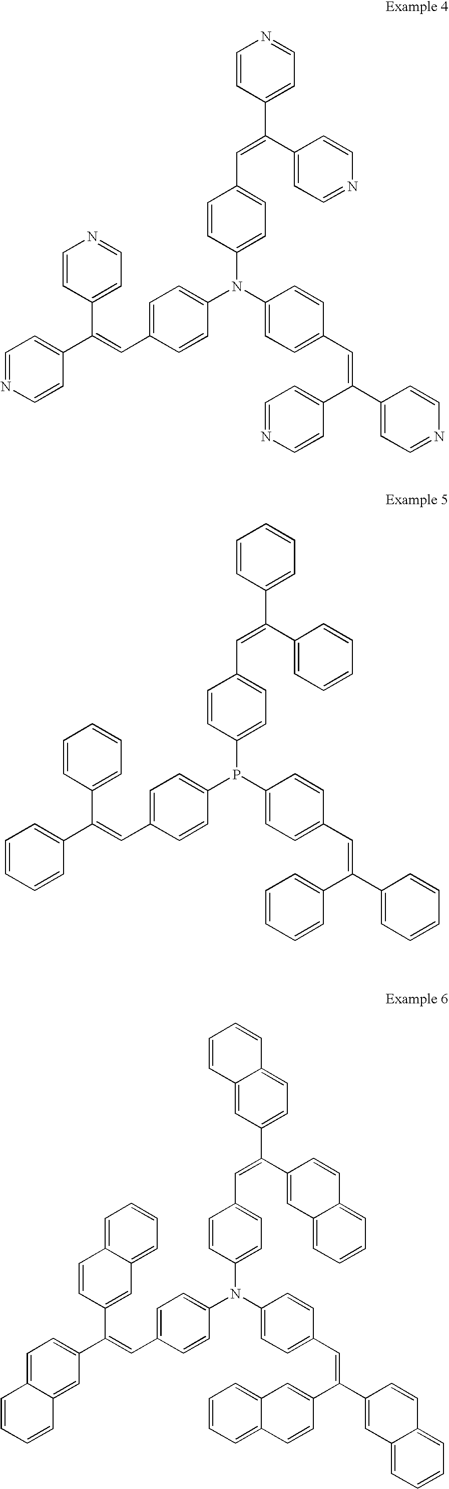

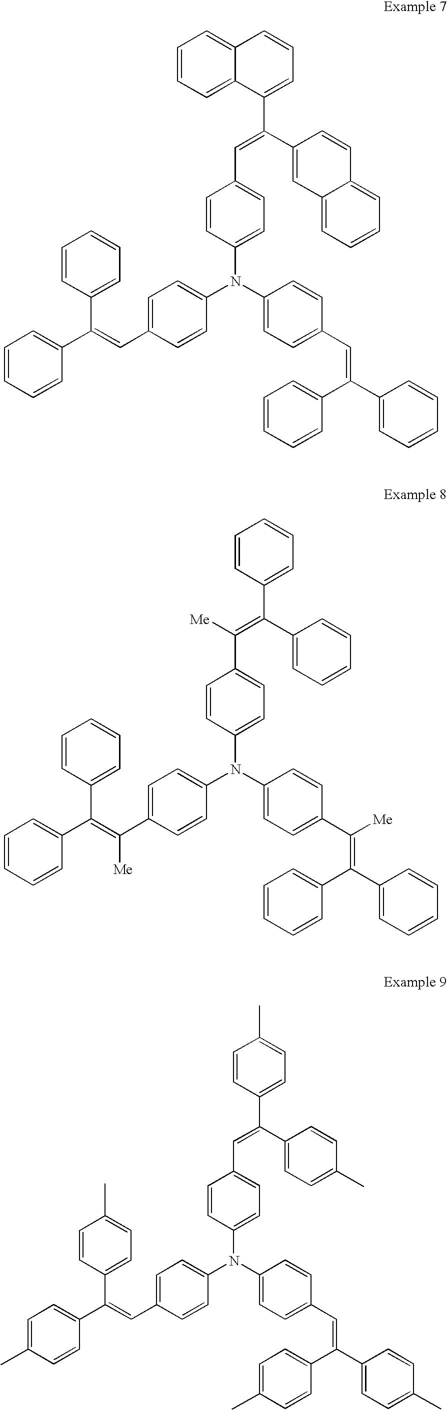

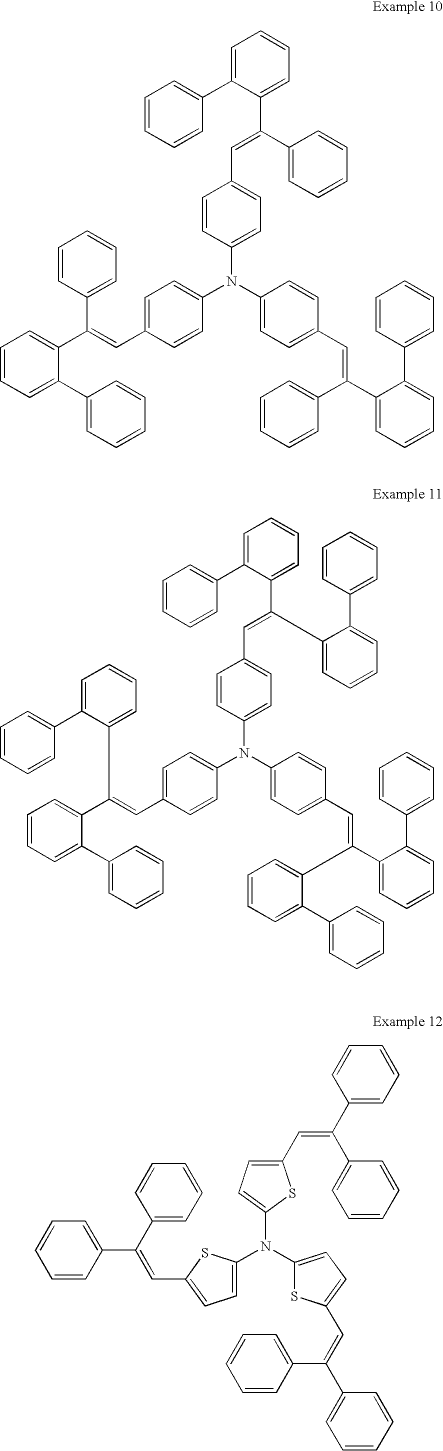

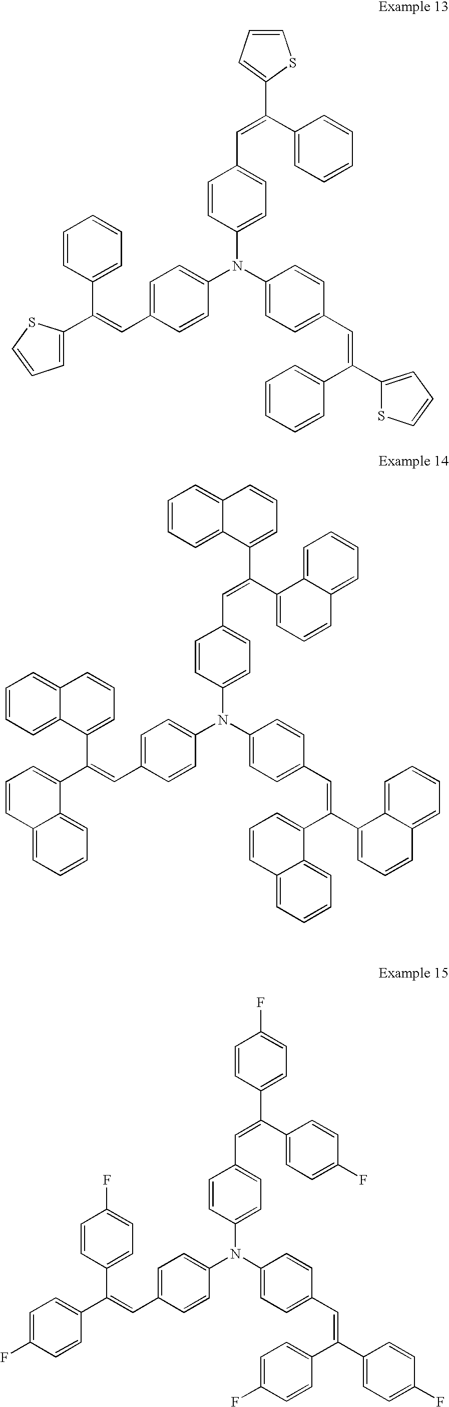

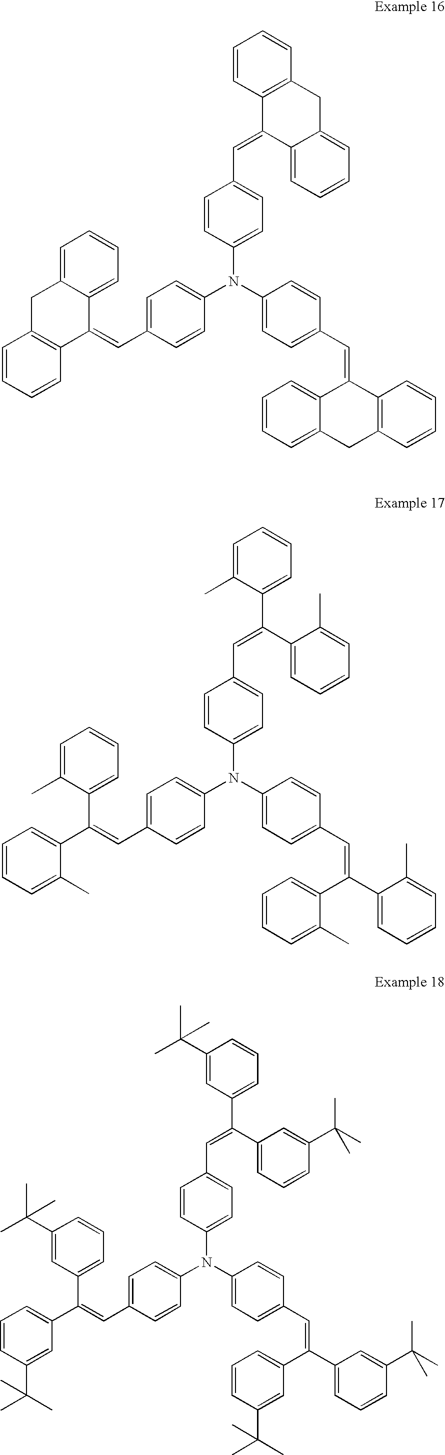

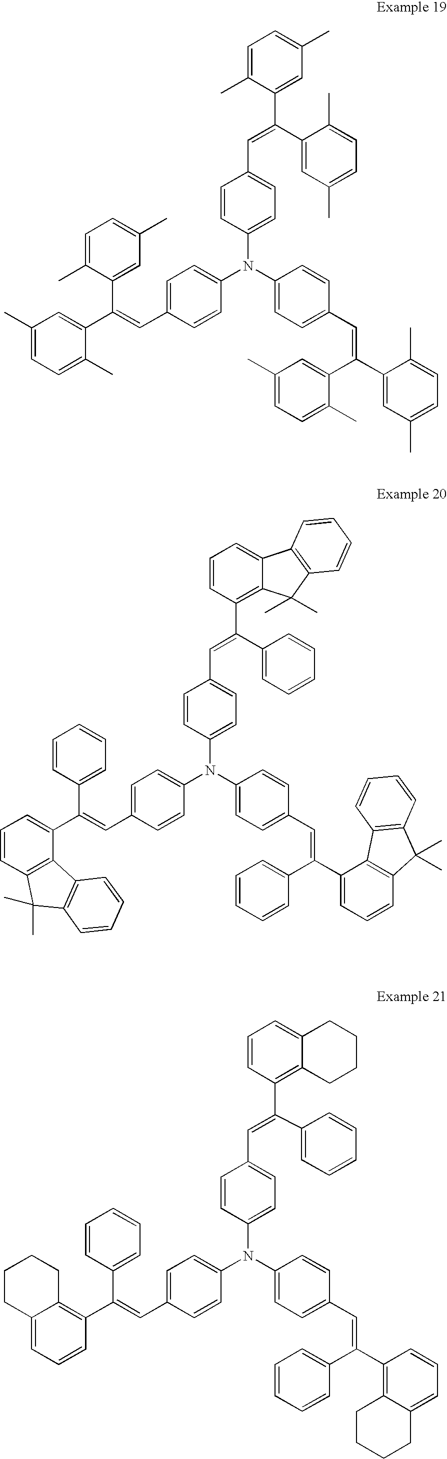

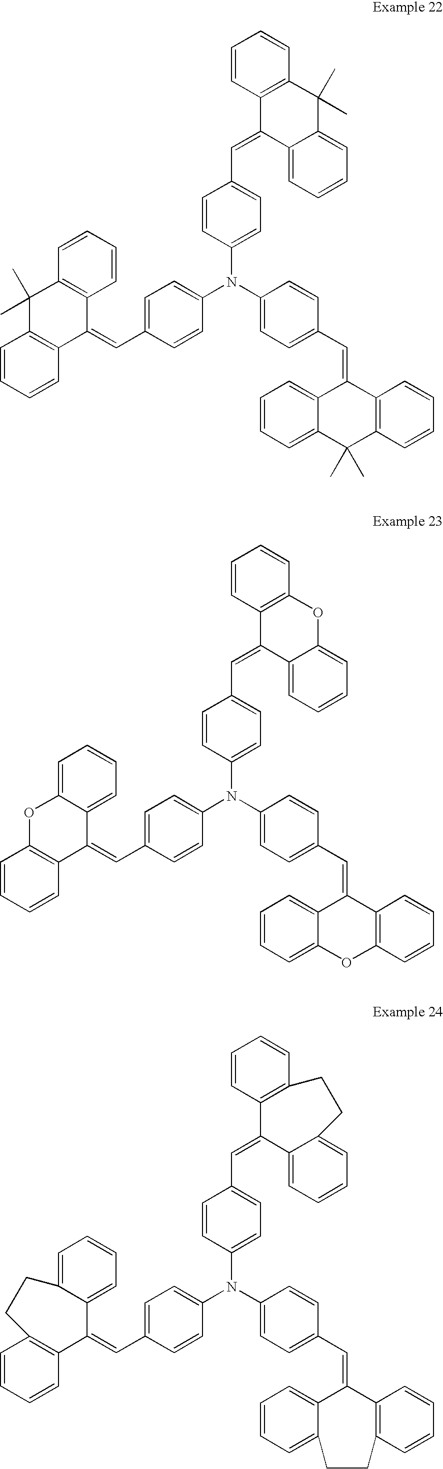

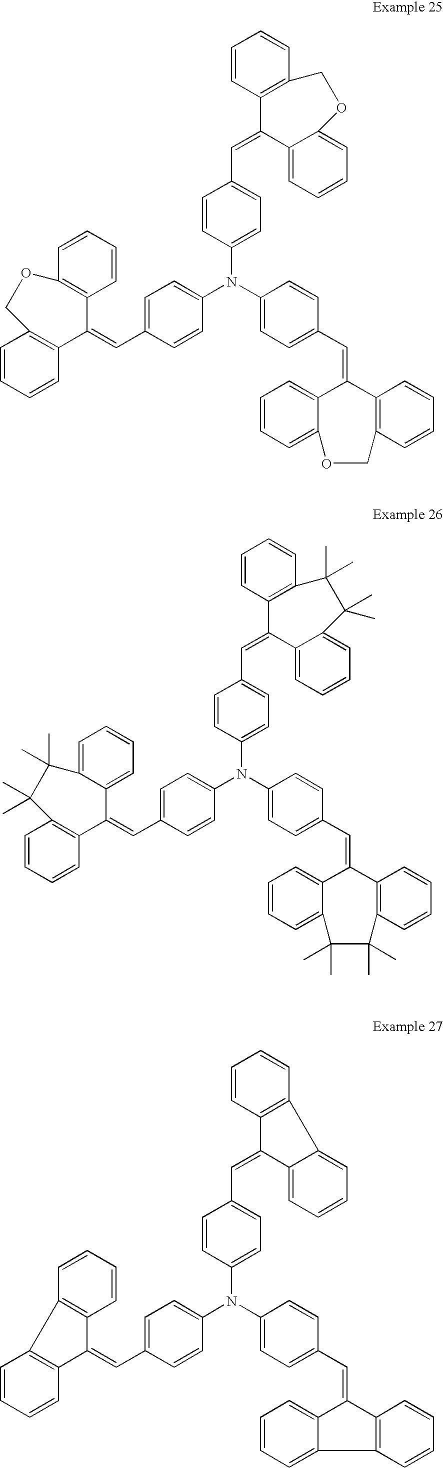

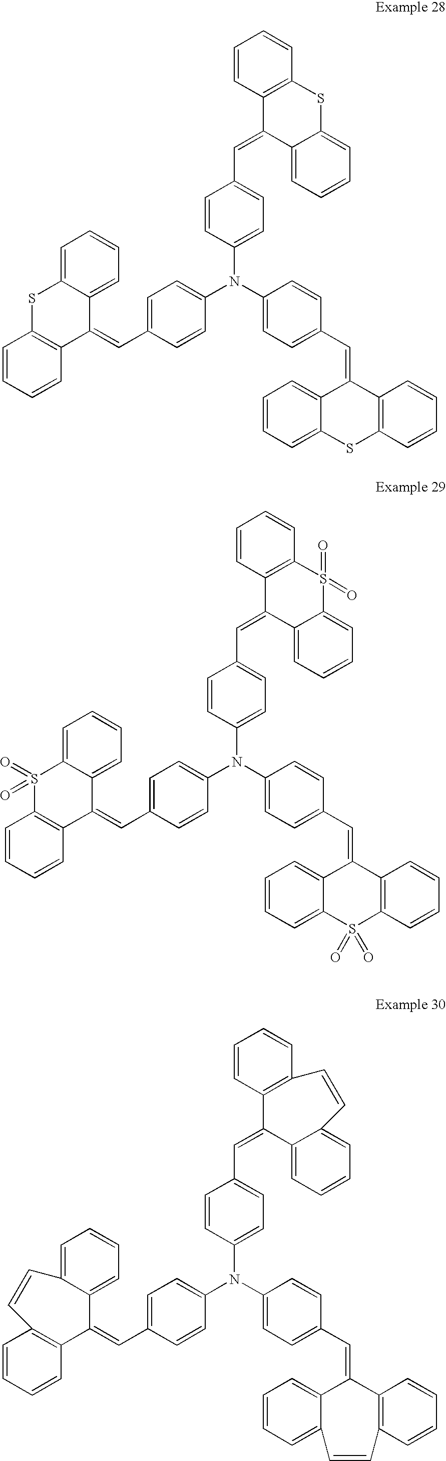

Compounds of the formula (1) in which the symbols Y and Z each, identically or differently on each occurrence, stand for an aryl or heteroaryl group and form a ring system with one another via a substituent R¹ or via a direct bond are novel and are therefore likewise a subject-matter of the invention.

The invention furthermore relates to compounds of the formula (2)

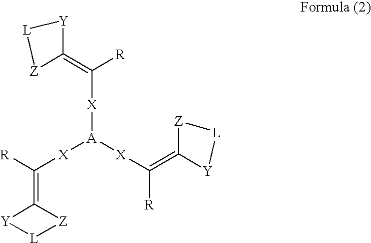

where the symbols A, X, R, R¹ and R² have the same meaning as described above and furthermore:
Y, Z are on each occurrence, identically or differently, an aryl or heteroaryl group having 2 to 40 C atoms, which may be substituted by one or more radicals R¹;
L is on each occurrence, identically or differently, a covalent bond or a divalent bridge having 1, 2, 3, 4 or 5 bridge atoms.

In a preferred embodiment, Y and Z stand for the same aryl or heteroaryl group, very particularly preferably for benzene.

L furthermore preferably stands for a covalent bond or a divalent bridge, selected from C(R¹)₂, O, S, SO, SO₂, N(R¹), P(R¹), PO(R¹), C(R¹)₂—C(R¹)₂, C(R¹)₂—O, C(R¹)₂—N(R¹), C(R¹)₂—C(R¹)₂—C(R¹)₂ and C(R¹)₂—O—C(R¹)₂. L particularly preferably stands for a divalent bridge selected from C(R¹)₂ and C(R¹)₂—C(R¹)₂.

The compound of the formula (1) or of the formula (2) is employed in the emitting layer as a mixture with at least one host material. It is preferred here for the compound of the formula (1) or of the formula (2) to be the emitting compound (the dopant) in the mixture and for the host material only to emit to a lesser extent or not at all.

Preferred host materials are organic compounds whose emission is of shorter wavelength than that of the compound of the formula (1) or of the formula (2) or which do not emit at all in the visible region.

Suitable host materials are various classes of substance. Preferred host materials are selected from the classes of the oligoarylenes (for example 2, 2′,7,7′-tetraphenylspirobifluorene as described in EP 676461 or dinaphthylanthracene), the oligoarylenevinylenes (for example DPVBi or spiro-DPVBi as described in EP 676461), the polypodal metal complexes (for example as described in WO 04/081017), the hole-conducting compounds (for example as described in WO 04/058911) or the electron-conducting compounds, in particular ketones, phosphine oxides, sulfoxides, etc. (for example as described in the as yet unpublished patent application DE 102004008304.5). Particularly preferred host materials are selected from the classes of the oligoarylenes comprising naphthalene, anthracene and/or pyrene, the oligoarylenevinylenes, the ketones, the phosphine oxides and the sulfoxides.

The proportion of the compound of the formula (1) or of the formula (2) in the mixture is between 0.1 and 99% by weight, preferably between 0.5 and 50% by weight, particularly preferably between 1 and 20% by weight, especially between 1 and 10% by weight.

Correspondingly, the proportion of the host material in the mixture is between 1 and 99.9% by weight, preferably between 50 and 99.5% by weight, particularly preferably between 80 and 99% by weight, especially between 90 and 99% by weight.

Preference is furthermore given to organic electroluminescent devices, characterised in that a plurality of emitting compounds are used in the same layer or in different layers, where at least one of these compounds has a structure of the formula (1) or of the formula (2). These compounds particularly preferably have in total a plurality of emission maxima between 380 nm and 750 nm, resulting overall in white emission, i.e. at least one further emitting compound which is able to fluoresce or phosphoresce and emits yellow, orange or red light is used in addition to the compound of the formula (1) or of the formula (2). A structure of this type is described, for example, in WO 05/011013.

Apart from the emitting layer, the organic electroluminescent device may also comprise further layers. These can be, for example: hole-injection layer, hole-transport layer, electron-transport layer and/or electron-injection layer. However, it should be pointed out at this point that each of these layers does not necessarily have to be present.

Thus, in particular in the case of the use of compounds of the formula (1) or of the formula (2) with electron-conducting host materials, very good results are furthermore obtained if the organic electroluminescent device does not comprise a separate electron-transport layer and the emitting layer is directly adjacent to the electron-injection layer or to the cathode. It may likewise be preferred if the organic electroluminescent device does not comprise a separate hole-transport layer and the emitting layer is directly adjacent to the hole-injection layer or to the anode. It may furthermore be preferred if the compound of the formula (1) or of the formula (2) is not used only as dopant in the emitting layer, but also in addition as hole-conducting compound (as the pure substance or as a mixture) in the hole-transport layer. Preferred materials for the electron-transport layer are metal complexes comprising aluminium or gallium, polypodal metal complexes (for example as described in WO 04/081017), ketones, phosphine oxides or sulfoxides (for example as described in the as yet unpublished patent application DE 102004008304.5). Particularly preferred materials for the electron-transport layer are ketones and phosphine oxides.

Preference is furthermore given to an organic electroluminescent device, characterised in that one or more layers are coated by means of a sublimation method, in which the materials are vapour-deposited in vacuum sublimation units at a pressure below 10⁻⁵ mbar, preferably below 10⁻⁶ mbar, particularly preferably below 10⁻⁷ mbar.

Preference is likewise given to an organic electroluminescent device, characterised in that one or more layers are coated by means of the OVPD (organic vapour phase deposition) method or with the aid of carrier-gas sublimation, where the materials are applied at a pressure between 10⁻⁵ mbar and 1 bar.

Preference is furthermore given to an organic electroluminescent device, characterised in that one or more layers are produced from solution, such as, for example, by spin coating, or by means of any desired printing process, such as, for example, screen printing, flexographic printing or offset printing, but particularly preferably LITI (light induced thermal imaging, thermal transfer printing) or ink-jet printing.

The emitting devices described above have the following surprising advantages over the prior art:
1. The efficiency of corresponding devices becomes higher compared with systems in accordance with the prior art.
2. The stability of corresponding devices becomes higher compared with systems in accordance with the prior art, which is evident, in particular, from a significantly longer lifetime.
3. The operating voltage is reduced. The power efficiency is consequently increased. This applies, in particular, if a phosphine oxide is used as host material.
4. The compounds can be sublimed and vapour-deposited well and without considerable decomposition, are consequently easier to process and are therefore more suitable for use in OLEDs than materials in accordance with the prior art, in particular than materials containing stilbene units which are not further substituted at the double bond.

The invention is explained in greater detail by the following examples without wishing it to be restricted thereby.

### EXAMPLES

### Example 1

### Synthesis of Dopant D1

The following synthesis was carried out as described in WO 02/10093 and, unless indicated otherwise, under a protective-gas atmosphere. The starting materials were purchased from ALDRICH (solvents, palladium(II) acetate, dimethylglycine, iron(III) chloride, tris(4-bromophenyl)amine, 1,1-diphenylethene).

A mixture of 24.1 g (50 mmol) of tris(4-bromophenyl)amine, 39.7 ml (225 mmol) of 1,1-diphenylethene, 337 mg (1.5 mmol) of palladium(II) acetate, 1.55 g (15 mmol) of N,N-dimethylglycine, 243 mg (1.5 mmol) of iron(III) chloride and 37.8 g (450 mmol) of sodium hydrogencarbonate in 500 ml of NMP was slowly heated to 140° C. with vigorous stirring and subsequently stirred at this temperature for 16 h. After cooling, 500 ml of dichloromethane and 1000 ml of water were added. The organic phase was separated off and washed five times with 500 ml of water. After drying over sodium sulfate, the organic phase was evaporated to dryness, the resultant yellow solid was recrystallised four times from DMSO, then washed by stirring twice with 500 ml of ethanol under reflux and subsequently sublimed three times under reduced pressure (T=315° C., p=5×10⁻⁵ mbar), giving 33.5 g (corresponding to 85.9% of theory) of the product having a purity of greater than 99.9% according to HPLC.

¹H-NMR (CDCl₃): δ [ppm]=7.35-7.21 (m, 30H, C₆H₅), 6.88 (s, 3H, vinyl-CH), 6.83, 6.75 (d, ³J_{HH}=8.7 Hz, 12H, C₆H₄).

### Example 2

### Production of OLEDs

OLEDs were produced by a general process as described in WO 04/058911, which was adapted in the individual case to the particular circumstances (for example layer-thickness variation in order to achieve optimum efficiency or colour).

The results for various OLEDs are presented in Examples 3 to 7 below. The basic structure, the materials and layer thicknesses used, apart from the emitting layer and the electron-transport layer, were identical in Examples 3 to 6 for better comparability. OLEDs having the following structure were produced analogously to the above-mentioned general process (where the hole-transport layer indicated below does not apply to Example 7):
Hole-injection layer (HIL) 60 nm PEDOT (spin-coated from water; purchased from H. C. Starck, Goslar, Germany; poly(3,4-ethylenedioxy-2,5-thiophene))
Hole-transport layer (HTM) 20 nm NaphDATA (vapour-deposited; purchased from SynTec, Wolfen, Germany; 4,4′,4″-tris(N-1-naphthyl-N-phenylamino)triphenylamine)
Hole-transport layer (HTM) 20 nm S-TAD (vapour-deposited; prepared as described in WO 99/12888; 2,2′,7,7′-tetrakis(diphenylamino)spiro-9,9′-bifluorene)
Emission layer (EML) see Table 1 for materials, concentration and layer thicknesses
Electron conductor (ETL) see Table 1 for materials and layer thicknesses
Ba/Al (cathode) 3 nm Ba, 150 nm Al on top

In Example 7, the hole-transport materials used, apart from those mentioned above, were NPB (N-naphthyl-N-phenyl-4,4′-diaminobiphenyl) and the compound HTM1 (2,2′,7,7′-tetrakis(di-para-tolylamino)spiro-9,9′-bifluorene).

These as yet unoptimised OLEDs were characterised by standard methods; for this purpose, the electroluminescence spectra, the efficiency (measured in cd/A), the power efficiency (measured in Im/W) as a function of the brightness, calculated from current/voltage/brightness characteristic lines (IUL characteristic lines), and the lifetime were determined. The lifetime is defined as the time after which the initial brightness of the OLED has dropped to half at a constant current density of 10 mA/cm².

Table 1 shows the results for some OLEDs (Examples 3 to 6), with the composition of the EML and ETL, including the layer thicknesses, also being shown in each case. The EMLs comprise dopant D1 (as described in Example 1) as emitting materials of the formula (1). OLEDs which comprise, as emitting compounds, dopant D2 in accordance with the above-mentioned prior art or only the host material serve as comparative examples. Compounds H1 to H4 depicted below serve as host materials. ETM1 (AlQ₃, purchased from SynTec, tris(quinolinato)aluminium(III)), ETM2 (bis(9,9′-spirobifluoren-2-yl) ketone as described in WO 04/093207) and ETM3 (bis(9,9′-spirobifluoren-2-yl)phenylphosphine oxide as described in WO 05/003253) serve as electron-transport materials.

Table 2 shows the results for further OLEDs (Example 7) which have been optimised for better efficiency and lifetime by variation of the hole-transport layers. For better clarity, the corresponding structural formulae of the dopants and host materials used are depicted below:

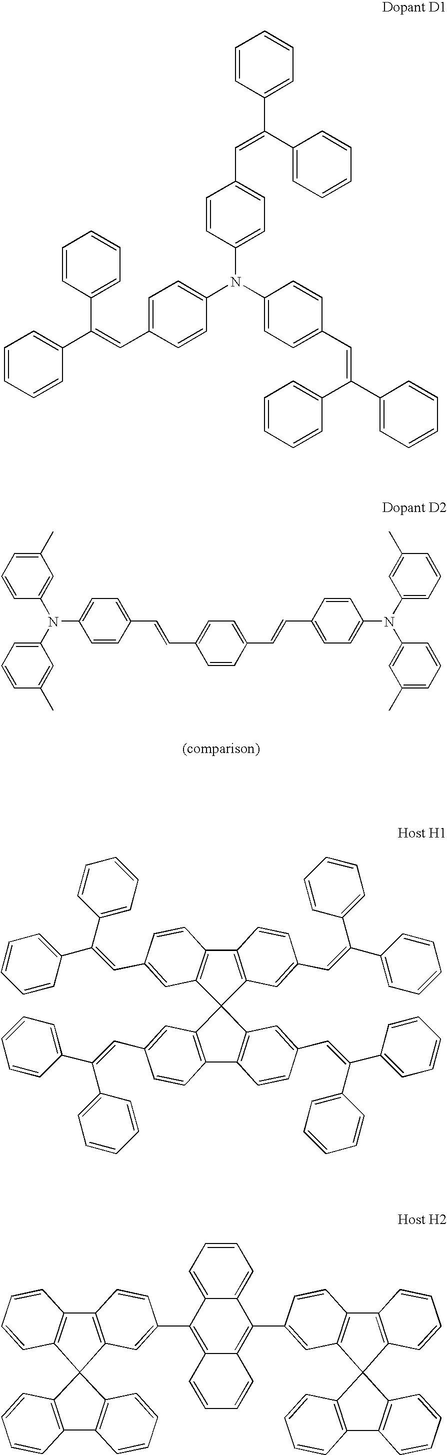

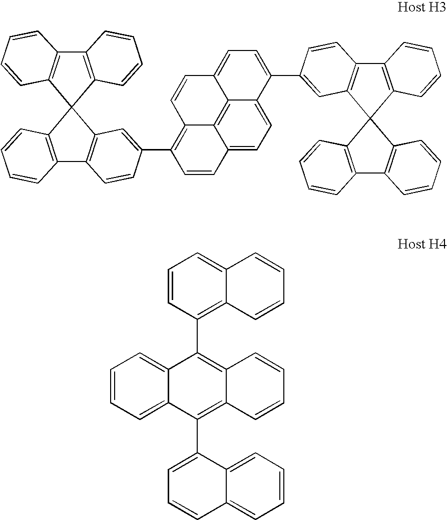

TABLE 1

| | | | Max. efficiency | Voltage | | |
|---|---|---|---|---|---|---|
| Example | EML | ETL | (cd/A) | (V) at 100 cd/m² | CIE | Lifetime (h) |
| Example 3a | H1 | ETM1 | 4.2 | 5.8 | x = 0.17; y = 0.26 | 1200 |
| (comparison) | (30 nm) | (20 nm) | | | | |
| Example 3b | H1:D2 (5%) | ETM1 | 4.9 | 6.3 | x = 0.17; y = 0.31 | 1000 |
| (comparison) | (30 nm) | (20 nm) | | | | |
| Example 3c | H1:D1 (2%) | ETM1 | 4.5 | 5.4 | x = 0.17; y = 0.30 | 2200 |
| | (30 nm) | (20 nm) | | | | |
| Example 3d | H1:D1 (5%) | ETM1 | 4.8 | 5.2 | x = 0.18; y = 0.33 | 4000 |
| | (30 nm) | (20 nm) | | | | |
| Example 3e | H1:D1 (5%) | ETM2 | 4.5 | 4.8 | x = 0.17; y = 0.29 | 4500 |
| | (30 nm) | (20 nm) | | | | |
| Example 3f | H1:D1 (5%) | ETM2 | 5.0 | 5.0 | x = 0.17; y = 0.30 | 3800 |
| | (30 nm) | (30 nm) | | | | |
| Example 3g | H1:D1 (5%) | ETM2 | 5.2 | 5.2 | x = 0.17; y = 0.31 | 3600 |
| | (30 nm) | (40 nm) | | | | |
| Example 4a | H2 | ETM1 | 3.3 | 6.5 | x = 0.15; y = 0.15 | 700 |
| (comparison) | (30 nm) | (20 nm) | | | | |
| Example 4b | H2:D1 (2%) | ETM1 | 4.5 | 5.7 | x = 0.17; y = 0.25 | 1800 |
| | (30 nm) | (20 nm) | | | | |
| Example 4c | H2:D1 (5%) | ETM1 | 4.8 | 5.3 | x = 0.18; y = 0.30 | 3800 |
| | (30 nm) | (20 nm) | | | | |
| Example 5a | H3 | ETM1 | 3.7 | 5.8 | x = 0.17; y = 0.26 | 1400 |
| (comparison) | (30 nm) | (20 nm) | | | | |
| Example 5b | H3:D2 (5%) | ETM1 | 5.4 | 6.5 | x = 0.19; y = 0.37 | 1000 |
| (comparison) | (30 nm) | (20 nm) | | | | |
| Example 5c | H3:D1 (2%) | ETM1 | 4.7 | 5.7 | x = 0.16; y = 0.28 | 2900 |
| | (30 nm) | (20 nm) | | | | |
| Example 5d | H3:D1 (5%) | ETM1 | 5.5 | 5.5 | x = 0.17; y = 0.31 | 5400 |
| | (30 nm) | (20 nm) | | | | |
| Example 6a | H4 | ETM1 | 1.1 | 5.8 | x = 0.17; y = 0.19 | 3100 |
| (comparison) | (30 nm) | (20 nm) | | | | |
| Example 6b | H4:D1 (2%) | ETM1 | 6.5 | 5.7 | x = 0.16; y = 0.28 | 7000 |
| | (30 nm) | (20 nm) | | | | |
| Example 6c | H4:D1 (5%) | ETM1 | 7.9 | 5.3 | x = 0.17; y = 0.31 | 15000 |
| | (30 nm) | (20 nm) | | | | |
| Example 6d | H4:D1 (5%) | ETM2 | 6.5 | 5.0 | x = 0.17; y = 0.28 | 16500 |
| | (30 nm) | (20 nm) | | | | |
| Example 6e | H4:D1 (5%) | ETM2 | 6.7 | 5.2 | x = 0.17; y = 0.31 | 15000 |
| | (30 nm) | (30 nm) | | | | |
| Example 6f | H4:D1 (5%) | ETM2 | 7.4 | 5.4 | x = 0.17; y = 0.32 | 14000 |
| | (30 nm) | (40 nm) | | | | |
| Example 6g | H4:D1 (5%) | ETM3 | 4.7 | 5.1 | x = 0.17; y = 0.30 | 20500 |
| | (30 nm) | (20 nm) | | | | |
| Example 6h | H4:D1 (5%) | ETM3 | 4.9 | 5.4 | x = 0.17; y = 0.31 | 20000 |
| | (30 nm) | (30 nm) | | | | |

TABLE 2

| | | | | | Max. efficiency | Voltage | | |
|---|---|---|---|---|---|---|---|---|
| Example | HTL1 | HTL2 | EML | ETL | (cd/A) | (V) at 100 cd/m² | CIE | Lifetime (h) |
| Example 7a | HTM1 | S-TAD | H4:D1 (5%) | ETM1 | 7.6 | 4.4 | x = 0.17; y = 0.28 | 17000 |
| | (20 nm) | (20 nm) | (30 nm) | (20 nm) | | | | |
| Example 7b | NaphDATA | NPB | H4:D1 (5%) | ETM1 | 10.5 | 5.3 | x = 0.17; y = 0.28 | 15500 |
| | (20 nm) | (20 nm) | (30 nm) | (20 nm) | | | | |
| Example 7c | HTM1 | NPB | H4:D1 (5%) | ETM1 | 8.1 | 4.8 | x = 0.17; y = 0.28 | 20000 |
| | (20 nm) | (20 nm) | (30 nm) | (20 nm) | | | | |

### Example 8

### Comparison of the Thermal Stability

As described in Example 1, dopant D1 (molecular weight=780 g/mol) was sublimed three times at 315° C. under reduced pressure. The sublimation left no residue and no indication of decomposition, checked by ¹H-NMR and HPLC. Storage experiments at 330° C. for 160 h likewise gave no indication of thermally induced decomposition of dopant D1.

By comparison, dopant D2 (comparative example in accordance with the prior art, molecular weight=673 g/mol) has significantly lower stability. Sublimation of D2 having an initial purity, according to ¹H-NMR and HPLC, of greater than 99.9% at T=330° C. and p=5×10⁻⁵ mbar gave after 2 h:
about 69% by weight of the amount employed in the form of a resinified residue which is only sparingly soluble in common organic solvents,
about 30% by weight of a yellow sublimate,
traces of colourless bis(3-methylphenyl)amine, determined by ¹H-NMR.

The yellow sublimate was not homogeneous. According to ¹H-NMR and HPLC analysis, it consisted of a mixture of trans,trans, cis,trans and cis,cis configuration isomers of D2. Storage experiments at 330° C. for 160 h resulted in virtually complete decomposition (resinification) of dopant D2.

These results show that dopant D1 has excellent long-term stability and is therefore extremely suitable for industrial use. In particular, the long-term temperature stability of dopant D1 is significantly higher than that of comparative dopant D2 in accordance with the prior art.

In summary, it can be stated that OLEDs comprising emitting compounds of the formula (1) have significantly higher efficiency at comparable or lower voltages and with a significantly longer lifetime than materials in accordance with the prior art, as can easily be seen from Table 1. In addition, due to the higher thermal stability, they can be processed significantly more easily than materials in accordance with the prior art, as described in Example 8. These compounds are therefore more suitable for use in OLEDs than are materials in accordance with the prior art.

1-21. (canceled)

22. An organic electroluminescent device comprising cathode, anode and at least one emitting layer, wherein the emitting layer comprises
a) 0.1 to 99% by weight of at least one compound of the formula (1) 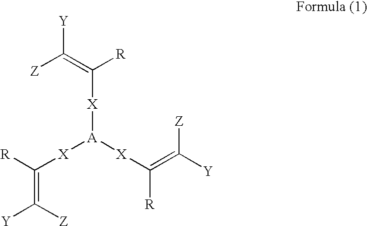
where the following applies to the symbols and indices:
A is on each occurrence nitrogen, phosphorus, P═O, P═S, arsenic, As═O, As═S, antimony, Sb═O or Sb═S;
X is on each occurrence, identically or differently, a divalent aromatic or heteroaromatic ring system having 2 to 60 C atoms, which is optionally substituted by one or more radicals R¹;
Y is on each occurrence, identically or differently, a monovalent aromatic or heteroaromatic ring system having 2 to 60 C atoms, which is optionally substituted by one or more radicals R¹, with the proviso that Y cannot contain substituted or unsubstituted amino groups;
Z is on each occurrence, identically or differently, Y or CN or a straight-chain, branched or cyclic alkyl group having 1 to 40 C atoms, which is optionally substituted by one or more radicals R², where one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms is optionally replaced by F, Cl, Br, I, CN or NO₂;
R is on each occurrence, identically or differently, H, CN or a straight-chain, branched or cyclic alkyl group having 1 to 40 C atoms, which is optionally substituted by one or more radicals R², where one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms is optionally replaced by F, Cl, Br, I, CN or NO₂;
R¹ is on each occurrence, identically or differently, H, F, Cl, Br, I, CN, NO₂, a straight-chain, branched or cyclic alkyl, alkoxy or thioalkoxy group having 1 to 40 C atoms, which is optionally substituted by one or more radicals R², where one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms is optionally replaced by F, Cl, Br, I, CN or NO₂, or an aromatic or heteroaromatic ring system, which is optionally substituted by one or more radicals R², or an aryloxy or heteroaryloxy group having 1 to 40 aromatic C atoms, which is optionally substituted by one or more radicals R², or a combination of two to five of these systems; two or more substituents R¹ here may also form a mono- or polycyclic, aliphatic or aromatic ring system with one another; with the proviso that R¹ does not represent a cyclohexylidenemethine group;
R² is on each occurrence, identically or differently, H or an aliphatic or aromatic hydrocarbon radical having 1 to 20 C atoms; and
b) 1 to 99.9% by weight of at least one host material.

23. The organic electroluminescent device according to claim 22, wherein A stands for nitrogen, phosphorus or P═O.

24. The organic electroluminescent device according to claim 22, wherein the symbol X, identically or differently on each occurrence, stands for a divalent aryl or heteroaryl group having 3 to 40 C atoms, which is optionally substituted by one to four radicals R.

25. The organic electroluminescent device according to claim 22, wherein the symbol Y, identically or differently on each occurrence, stands for a monovalent aryl or heteroaryl group having 3 to 40 C atoms, which is optionally substituted by one to four radicals R¹.

26. The organic electroluminescent device according to claim 22, wherein the symbol Z, identically or differently on each occurrence, is Y.

27. The organic electroluminescent device according to device according to claim 22, wherein the symbol R, identically or differently on each occurrence, is H or a methyl group.

28. The organic electroluminescent device according to device according to claim 22, wherein Y and Z in compounds of the formula (1), are bonded to an alkene group, each stand for the same monovalent aromatic ring system.

29. The organic electroluminescent devices according to claim 28, wherein all units Y and Z are selected identically, in compounds of the formula (1).

30. The organic electroluminescent device according to claim 22, wherein all units X are selected identically in compounds of the formula (1).

31. The organic electroluminescent device according to claim 30, wherein the compounds of the formula (1) have a symmetrical structure and C₃ symmetry.

32. The organic electroluminescent device according to claim 22, wherein the compounds of the formula (1) are selected from example structures 1 to 33

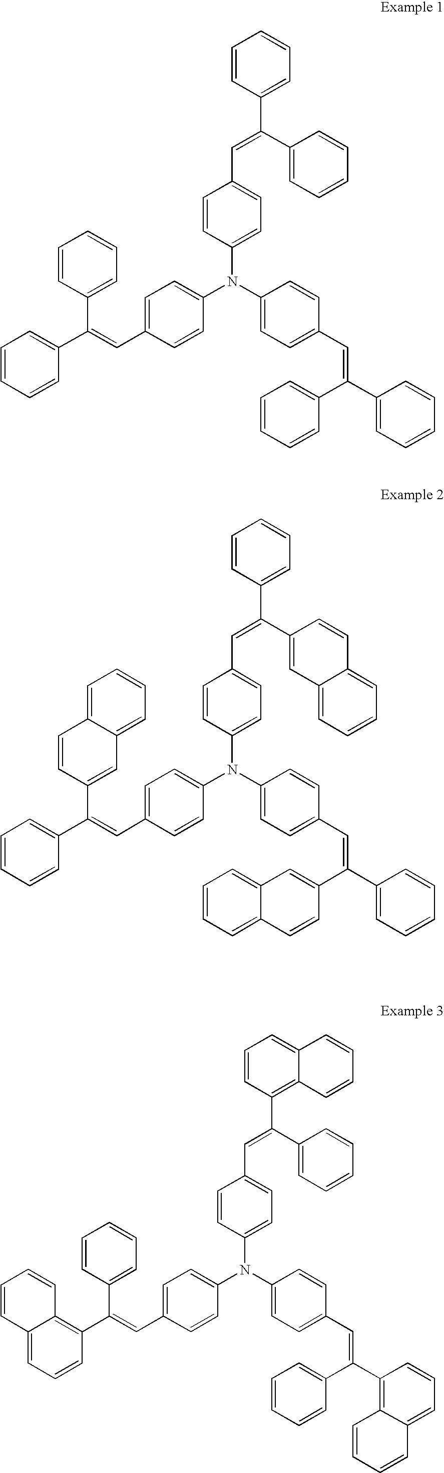

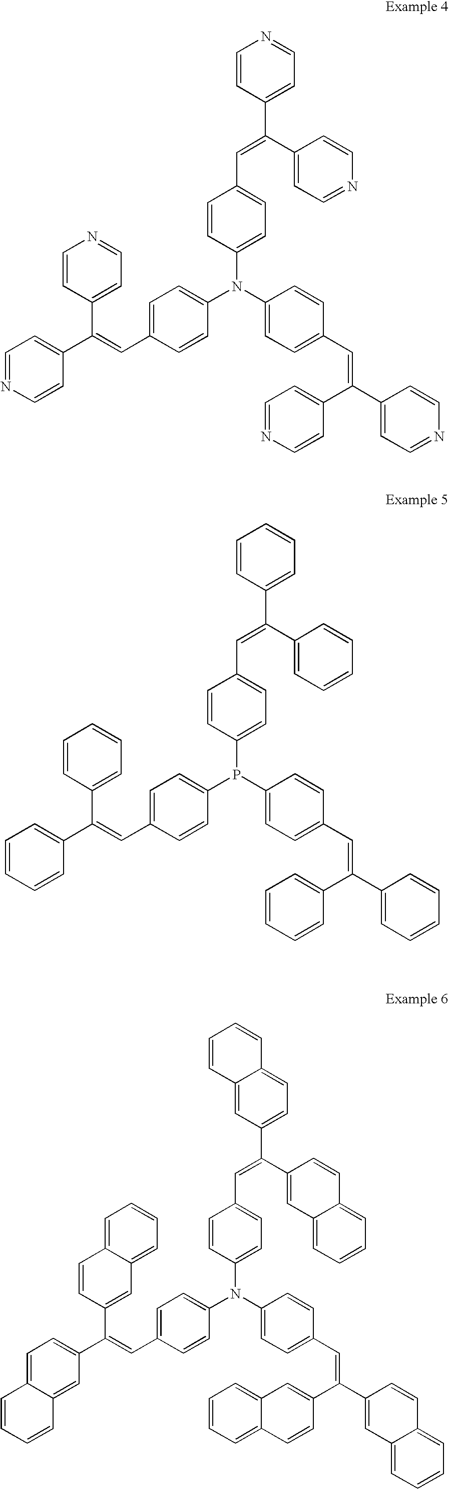

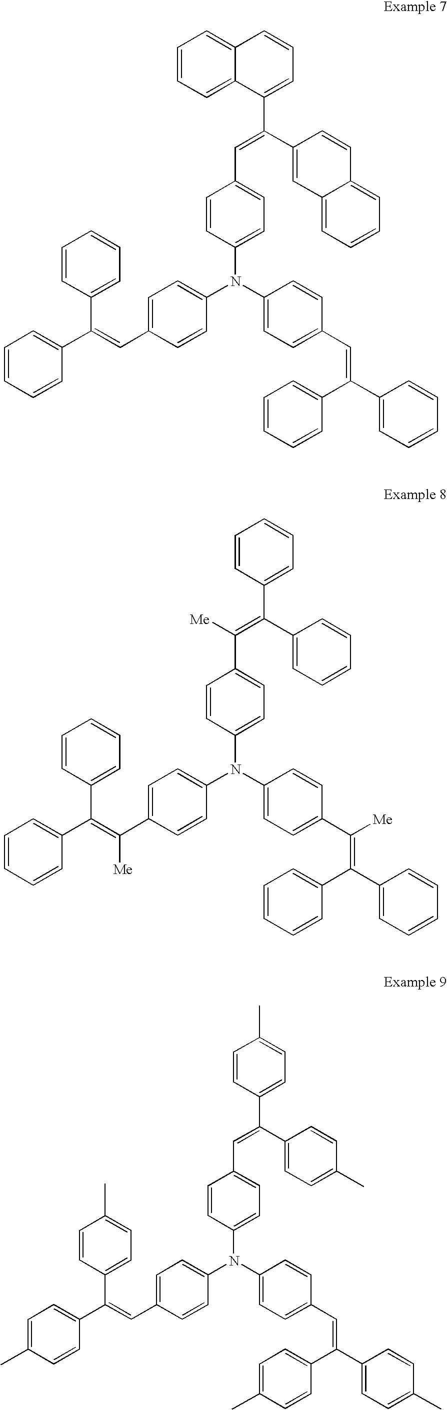

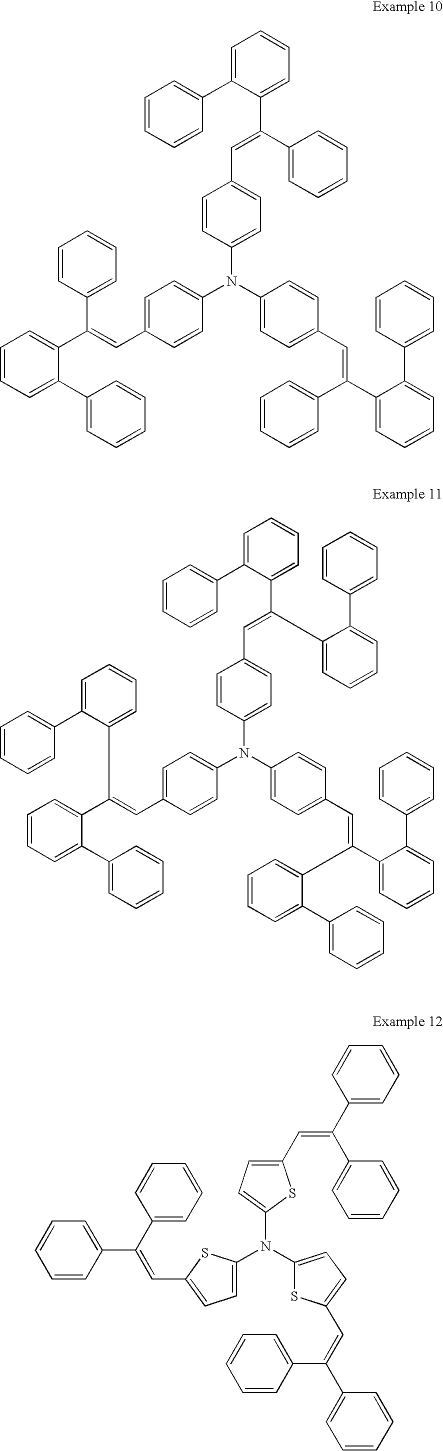

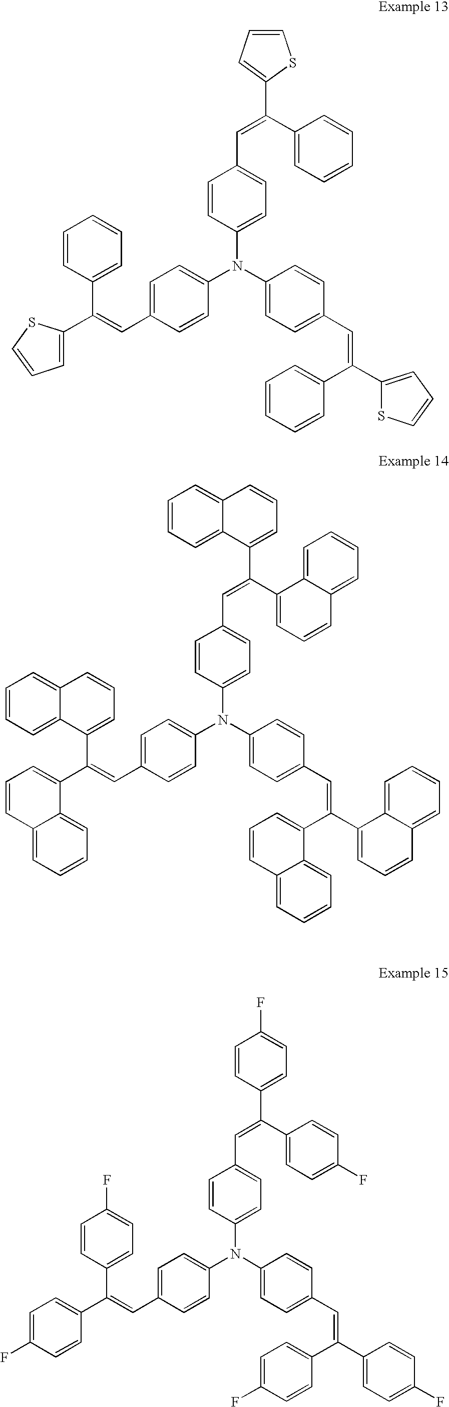

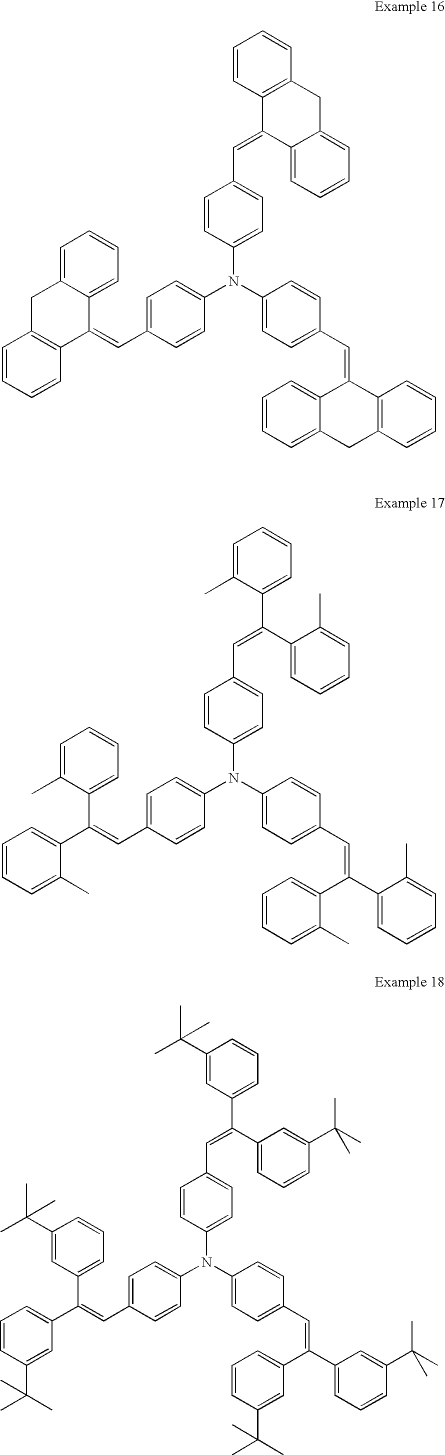

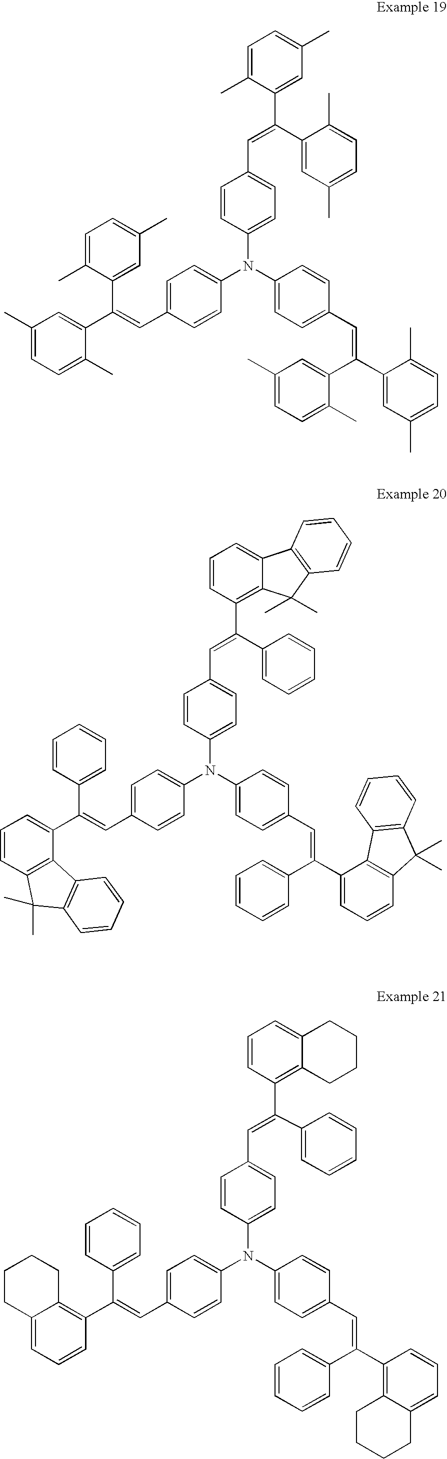

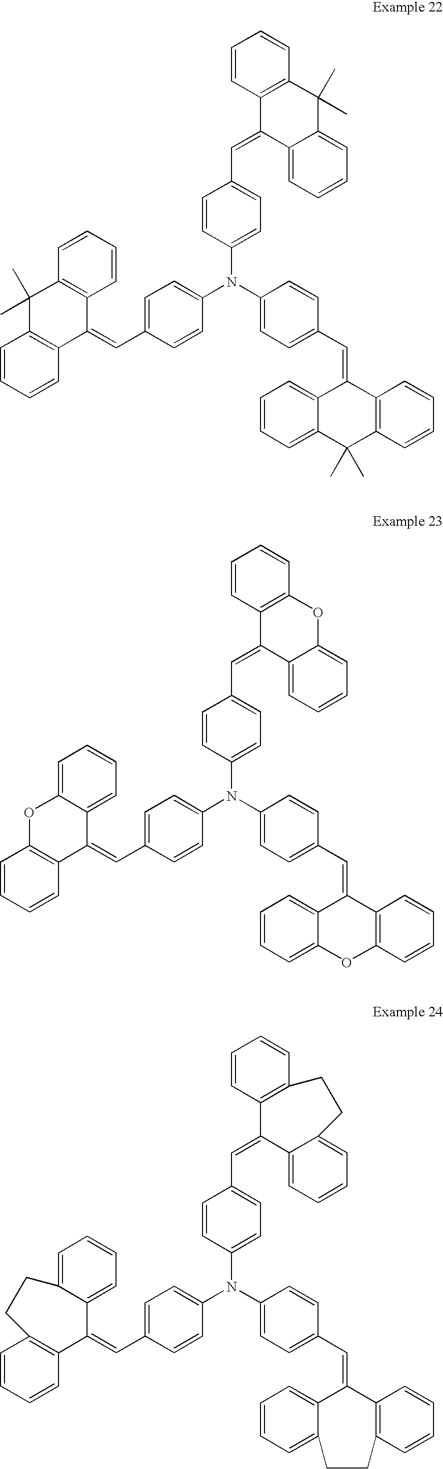

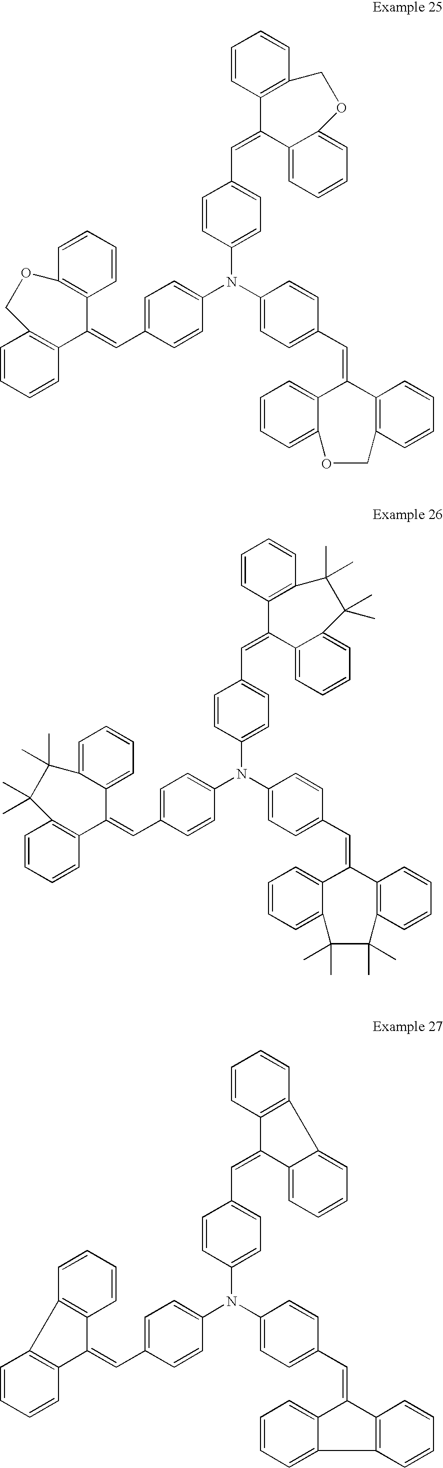

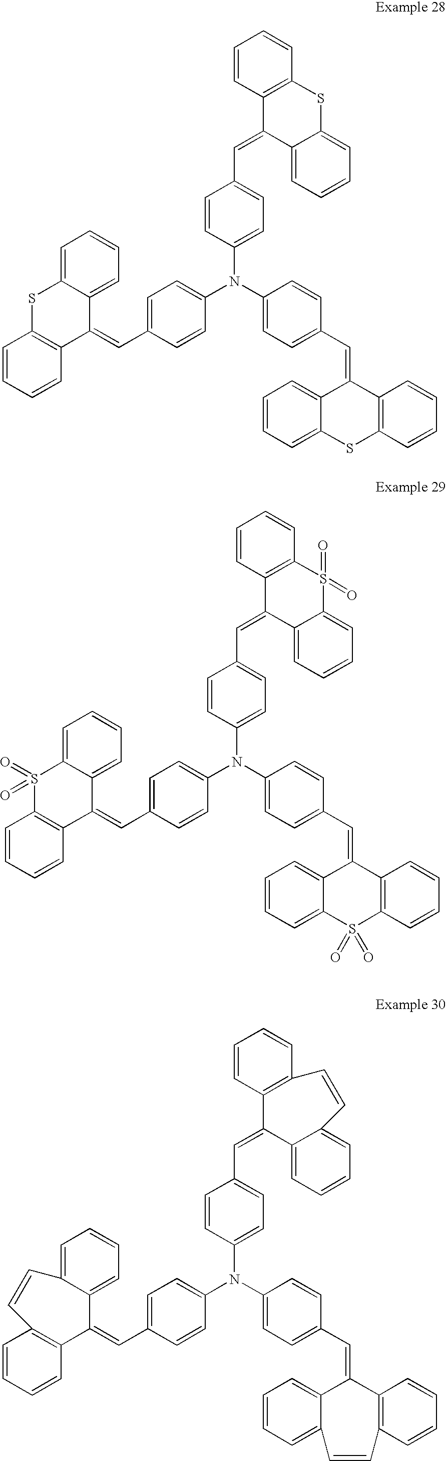

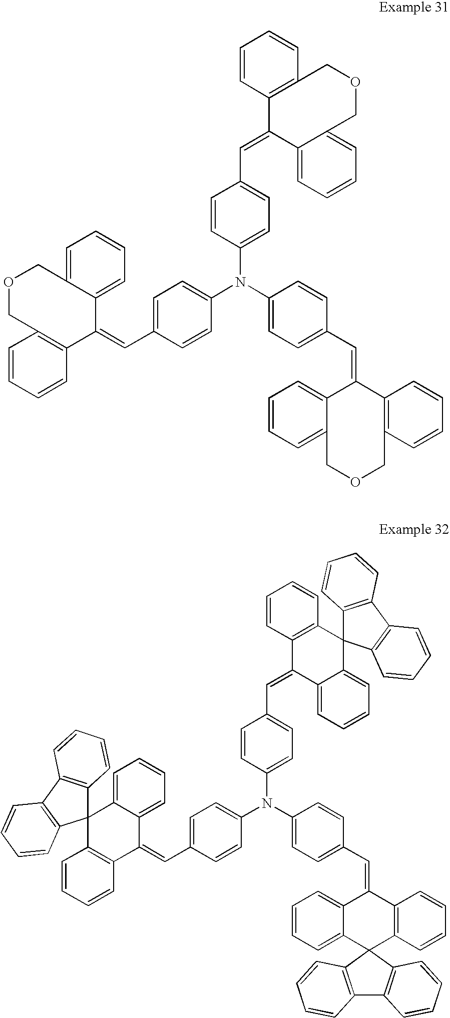

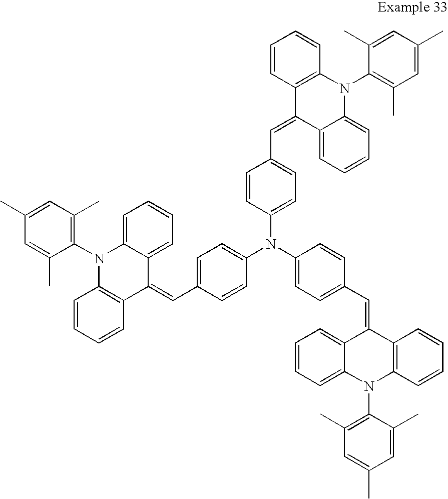

33. The organic electroluminescent device according to device according to claim 22, wherein the host materials are organic compounds whose emission is of shorter wavelength than that of the compound of the formula (1) or which do not emit at all in the visible region.

34. The organic electroluminescent device according to claim 22, wherein the host materials are selected from the classes of the oligoarylenes, the oligoarylenevinylenes, the polypodal metal complexes, the hole-conducting compounds and the electron-conducting compounds.

35. The organic electroluminescent device according to claim 34, wherein the host materials are selected from the classes of the oligoarylenes comprising naphthalene, anthracene and/or pyrene, the oligoarylenevinylenes, the ketones, the phosphine oxides and the sulfoxides.

36. The organic electroluminescent device according to claim 22, wherein the proportion of the compound of the formula (1) in the mixture is between 0.5 and 50% by weight.

37. The organic electroluminescent device according to claim 22, wherein a plurality of emitting compounds are used in the same layer or in different layers, where at least one of these compounds has a structure of the formula (1), and that these compounds have in total a plurality of emission maxima between 380 nm and 750 nm, resulting overall in white emission.

38. The organic electroluminescent device according to claim 22, wherein, apart from the emitting layer, one or more layers selected from hole-injection layers, hole-transport layers, electron-transport layers and/or electron-injection layers are also present.

39. The organic electroluminescent device according to claim 38, wherein at least one electron-transport layer comprising at least one keto compound, a phosphine oxide or a sulfoxide is present.

40. A compounds of the formula (2) 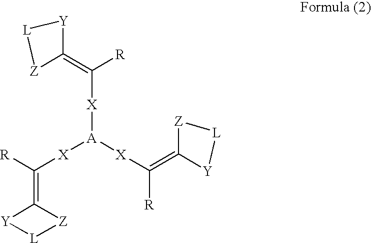
wherein
A is on each occurrence nitrogen, phosphorus, P═O, P═S, arsenic, As═O, As═S, antimony, Sb═O or Sb═S;
X is on each occurrence, identically or differently, a divalent aromatic or heteroaromatic ring system having 2 to 60 C atoms, which is optionally substituted by one or more radicals R¹;
R is on each occurrence, identically or differently, H, CN or a straight-chain, branched or cyclic alkyl group having 1 to 40 C atoms, which is optionally substituted by one or more radicals R², where one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms is optionally replaced by F, Cl, Br, I, CN or NO₂;
R¹ is on each occurrence, identically or differently, H, F, Cl, Br, I, CN, NO₂, a straight-chain, branched or cyclic alkyl, alkoxy or thioalkoxy group having 1 to 40 C atoms, which is optionally substituted by one or more radicals R², where one or more non-adjacent CH₂ groups is optionally replaced by —R²C═CR²—, —C≡C—, Si(R²)₂, Ge(R²)₂, Sn(R²)₂, C═O, C═S, C═Se, C═NR², —O—, —S— or —CONR²— and where one or more H atoms is optionally replaced by F, Cl, Br, I, CN or NO₂, or an aromatic or heteroaromatic ring system, which is optionally substituted by one or more radicals R², or an aryloxy or heteroaryloxy group having 1 to 40 aromatic C atoms, which is optionally substituted by one or more radicals R², or a combination of two to five of these systems; two or more substituents R¹ here may also form a mono- or polycyclic, aliphatic or aromatic ring system with one another; with the proviso that R¹ does not represent a cyclohexylidenemethine group;
R² is on each occurrence, identically or differently, H or an aliphatic or aromatic hydrocarbon radical having 1 to 20 C atoms;
Y and Z are on each occurrence, identically or differently, an aryl or heteroaryl group having 2 to 40 C atoms, which is optionally substituted by one or more radicals R¹; and
L is on each occurrence, identically or differently, a covalent bond or a divalent bridge having 1, 2, 3, 4 or 5 bridge atoms.

41. The compounds according to claim 41, wherein the symbols Y and Z stand for the same aryl or heteroaryl group.

42. The compounds according to claim 41, wherein L stands for a covalent bond or a divalent bridge, selected from C(R¹)₂, O, S, SO, SO₂, N(R¹), P(R¹), PO(R¹), C(R¹)₂—C(R¹)₂, C(R¹)₂—O, C(R¹)₂—N(R¹), C(R¹)₂—C(R¹)₂—C(R¹)₂ and C(R¹)₂—O—C(R¹)₂.
